(19) United States
(12) Patent Application Publication
(10) Pub. No.: US 2007/0259012 A1
(43) Pub. Date: Nov. 8, 2007

(54) Clear Cosmetic Compositions and Methods Of Use

(76) Inventors: John Castro, Huntington Station, NY (US); Garrett Vanderover, Belerose, NY (US); Milagro Serrano, Bayshore, NY (US); Tatyana Tabakman, Brooklyn, NY (US)

Correspondence Address: THE ESTEE LAUDER COS, INC, 155 PINELAWN ROAD, STE 345 S, MELVILLE, NY, 11747, US

(21) Appl. No.: 11/737,441

(22) Filed: Apr. 19, 2007

Related U.S. Application Data
(60) Provisional application No. 60/746,270, filed on May 3, 2006.

(51) Int. Cl. A61K 8/37

(52) U.S. Cl. 424/401

(57) ABSTRACT

A solid, substantially clear cosmetic composition having a refractive index of between 1.4-1.6 comprising a partially crystalline polyolefin homopolymer or copolymer wax and a solvent system, wherein the refractive index of the solvent system is matched with the refractive index of the wax. Also contemplated are methods of making the solid, substantially clear composition comprising the steps of (a) mixing the partially crystalline polyolefin homopolymer or copolymer wax within the solvent system and (b) cooling said composition to ambient temperature.

This application claims priority from U.S. 60/746270, filed May 3, 2006.

### FIELD OF THE INVENTION

The present invention relates to skin care cosmetic compositions and methods. In particular, the present invention relates to novel clear cosmetic composition comprising clear polypropylene and/or polyethylene waxes and methods of use thereof.

### BACKGROUND OF THE INVENTION

Traditional lipsticks often contain waxes, gellants, fillers, heavy oils and plasticizers for developing structure and creating a desired stiffness. Incorporation of waxes raises the melting point and therefore improves the physical stability of the composition. However, ingredients such as waxes that are used to provide the balance of rigidity and firmness to the structure have a disadvantage in that they contribute to opacity of the product. Therefore, there is a need to create clear lipsticks that have the desired balance of rigidity and firmness while remaining clear.

The art teaches various methods of creating stable, clear lipsticks. Polyamides have been used to help maintain clarity while achieving the desired stiffness in clear cosmetic compositions. For example, U.S. Pat. No. 3,148,125 discloses a clear lipstick in which the colorants are oil soluble or water soluble dyes, rather than pigments. The dyes are solubilized in a gel the base of which is polyamide resin. The water-soluble dyes are made compatible with the formulation by using lower aliphatic co-solvents.

However, polyamides exhibit various incompatibilities that make their use less than desirable. For example, polyamides are not compatible with a wide variety of oils, such as hydrocarbon oils, which are commonly used in cosmetic compositions. Moreover, polyamides are less tacky and therefore their adhesion properties are significantly impaired. As a result, polyamides are less desirable in products such as lipsticks which require minimally an initial phase of adhesion during the application of the product to the skin or hair. Furthermore, polyamides are difficult to use in compositions with high melting points such as lipsticks containing waxes.

Other substitutes for traditional waxes in lipsticks include dibutyl lauroyl glutamide (commercially available as G.P.-1), which is used to maintain clarity without compromising structure and stability. However, G.P.-1 used alone does not provide the desired amount of clarity without additional ingredients such as hydrocarbons, which at certain levels can affect the consistency of the composition by dissolving some of the existing wax. Gelling agents such as dibenzyl monosorbitol acetal, DMSA, have been used with lipophilic waxes to create substantially clear deodorant compositions. See U.S. Pat. No. 5,750,125.

Refractive index matching allows for better passage of light with less light scattering through compositions such as gel sticks. The resulting product therefore appears more clear or translucent as a packaged composition or when initially applied topically to the skin. However, such compositions are very difficult to formulate, especially for three component systems (active, solvent and gellant) in which refractive index matching is extremely difficult.

Therefore, there still remains a need for stable anhydrous lipstick compositions comprising waxes that do not destroy clarity.

### SUMMARY OF THE INVENTION

The present invention comprises a solid, substantially clear cosmetic composition having a refractive index of between 1.4-1.6 comprising a partially crystalline polyolefin homopolymer or copolymer wax and a solvent system, wherein the refractive index of the solvent system is matched with the refractive index of the wax.

The present invention further comprises a method of making the solid, substantially clear composition comprising the steps of (a) mixing the partially crystalline polyolefin homopolymer or copolymer wax within the solvent system and (b) cooling said composition to ambient temperature.

### DETAILED DESCRIPTION

Except in operating and comparative examples, or where otherwise explicitly indicated, all numbers in this description indicating amounts or ratios of material or conditions of reaction, physical properties of materials and/or use are to be understood as modified by the word “about.” All amounts are by weight of the final composition, unless otherwise specified.

The present invention is directed to substantially clear solid cosmetic compositions. “Clear” it is meant in the usual dictionary definition of this term. Thus, a clear stick, like glass, allows for ready viewing of objects behind it. By contrast, a translucent cosmetic stick, although allowing light to pass through, causes the light to be so scattered that it will be impossible to clearly identify objects behind the translucent stick. Opaque sticks do not permit the light to pass through. Thus, according to the present invention there is a distinction between, for example, “clear” and “translucent” cosmetic compositions.

By stable, it is meant that the structure of the solid composition is physically stable to have the desired stiffness to form a solid and the composition is chemically stable such that the oily components incorporated within the composition do not leak or migrate out of the composition.

More specifically, in the present invention, it has been surprisingly found that combining a specific wax with a solvent system such that the refractive index of the solvent system matches the refractive index of the wax results in a clear, stable composition having a refractive index of between 1.4-1.6.

### Wax

The wax of the present invention is a polyolefin wax modified with silane compounds and prepared using metallocene catalysts. More specifically, the polyolefin wax is a partially crystalline polyolefin homopolymer or copolymer wax modified free-radically with a silane compound and having a viscosity as measured at 170° C. of between 10 and 50,000 mPas and a heat of fusion >10 J/g, wherein the silane compound used for modification includes at least one olefin double bond and at least one alkoxy radical attached directly to silicon and wherein the polyolefin waxes used for modification have been prepared using a metallocene catalyst. The polyolefin wax of the present invention and the process of making such are provided in further detail in U.S. Patent Application No. 2005-0043455, which is incorporated by reference herein.

The partially crystalline polyolefin homopolymer or copolymer wax of the present invention has a refractive index of between 1.4 to 1.6. The refractive index of a material may be measured by using a refractometer and by employing conventional refractory techniques. A particularly preferred refractometer used in the present invention is Fisher Scientific's Abbe Refractometer Model No. 6182 (available from Fisher Scientific of Springfield, N.J.).

The preferred polyolefin wax of the present invention is a polypropylene wax (Commercially available under the Tradename Licocene PP 1602 from Clariant). The polyolefin wax is present in an amount of from 0.1% to 20%, preferably from 1% to 10% and most preferably from 2% to 5%.

### Solvent

The second essential element of the present invention is a solvent system. It has been surprisingly found that a solvent system that has a refractive index that matches the refractive index of the wax of the present invention surprisingly creates a substantially clear solid anhydrous composition while maintaining stability. Specifically, the refractive index of the solvent system is between 1.4-1.6. Simply matching the refractive index of the solvent with the wax, however, does not yield a solid composition, as provided in more detail in Example 2 below. For example, a high concentration of hydrocarbons in a solvent system may dissolve the wax and therefore destroy the structure of the solid composition.

The solvent of the present invention may be selected from any solvent used in cosmetic compositions as long as the solvent does not change the refractive index of the solvent system, as indicated above. Specifically, the solvent of the present invention is selected from but not limited to Tridecyl Trimellitate (Hatcol 200), Isododecane (Permethyl 99A), Isohexadecane (Permethyl 101A), Isoeicosane (Permethyl 102A), Polyisobutene (Permethyl 104A, 106A, Presperse), Isodecyl Neopentanoate (Dermol 105), Lauryl Lactate (Dermol LL), Ethylhexyl Palmitate (Dermol 816), Octyldodecyl Behenate (Dermol 2022), Isododecyl Ethylhexanoate (Dermol 334), Polyglyceryl-2 Diisostearate (Dermol DGDIS), Synthetic Jojoba Oil (Wickenol 139), Octyldodecyl Myristate (Wickenol 142), Isononyl Isononanoate (Wickenol 151), Isodecyl Isononanoate (Wickenol 152), Isotridecyl Isononanoate (Wickenol 153), Octyl Stearate (Wickenol 156), Dioctyl Adipate (Wickenol 159), Dioctyl Adipate/Octyl Palmitate/Octyl Stearate (Wickenol 161), Octyl Stearate/Octyl Palmitate/Dioctyl Adipate (Wickenol 163), Hexyl Laurate (Cetiol A, Cognis), Coco-Caprylate/Caprate (Cetiol LC, Cognis), Cetearyl Isononanoate (Cetiol SN, Cognis), Isocetyl Stearate (Cetiol G-16S, Cognis), Isopropyl Myristate (Liponate IPM), Isopropyl Palmitate( Liponate IPP), Propylene Glycol Dipelargonate, Cetyl Octanoate (Trivent OC-16), Tricaprylin (Trivent OC-G), Octyldodecyl Myristate (Bemel Ester NPDC), Isostearyl Neopentanoate (Ceraphyl 375/Dermol 185), Decyl Oleate (Ceraphyl 140), Isodecyl Oleate (Ceraphyl 140A), Octyldodecyl Stearoyl Stearate (Ceraphyl 847), Caprylic/Capric Triglyceride (Lexol GT-865, Liponate GC-K, Myritol 318), Hydroxy-terminated Polyurethane (Polyolprepolymer-14), Natural Squalane, Isostearyl Neopentanoate (Bernel Ester 2014), Octyldodecanol (Eutanol G), Oleyl Lactate (Pelemol OL), Oleyl Alcohol (Novol), Oleyl Oleate (Schercemol OLO), Glycereth-7 Triacetate (Pelemol G7A), Polyglyceryl-3 Diisostearate (Synton PAO 100), Pentaerythrityl Tetraisostearate (Crodamol PTIS), Triglycerine Diisostearate (Emerest 2452), Oleyl Lactate (Plurol Diisostearique), Diglycerol Diisostearate/IPDI Copolymer (Polyderm PPI-DGDIS), Ricinus Communis (Castor) Seed Oil (Castor Oil USP/Crystal O), Menthyl Anthranilate (Neo Heliopan MA), Ethylhexyl Salicylate (Neo Heliopan OS), Polyisobutylene (Indopol H-1900), 2-Ethylhexyl-4-methoxycinnamate (Parsol MCX/Escolol 557) and combinations thereof.

The solvents of the present invention may each be used alone or in any combination in the solvent system as long as the total in the composition is between 1% to 90%, preferably from 10% to 80% and most preferably from 30% to 50% by weight of the total composition.

### Gellant

The present invention optionally comprises a gellant. The gellant of the present invention is selected from traditional gellants that do not destroy the refractive index of the composition of the present invention. Specifically, the gellant is selected from but not limited to Dibutyl/Laurol Glutamide (G.P.-1), Steroyl Inulin (Rheopearl Series) (available from Tomen America Inc.), Ethyl Hexyl Hydrostearate/Triethylhexyl Trimellitate/C30-45 Olefin (Clearwax (LCW)), 12-Hydroxystearic Acid (Phoenix Chemical), Polyethylene at low concentrations below 1% (Newphase, Presperse, Alzo Int'l, Mineral and Pigment Solutions, Inc.), Ethylenediamine/Stearyl Dimmer Tallate Copolymer (Uniclear 100VG, Arizona Chemicals) Polyamide resin (Macromelt6212, Versamid 940, GAX 14-030, GAX 14-029 (all available from Cognis)), Hydrogenated Styrene/Isoprene Copolymer (Kraton G-1701), Styrene-Ethylene/Butylene-Styrene Black Copolymer (Kraton G-1650, Kraton G-1652, Kraton G-1702 (Lipo Chemicals)), and combinations thereof.

The gellant is used in an amount of from 0.1% to 50%, preferably from 5% to 15%, and most preferably from 6% to 10% by weight of the composition.

### Other Ingredients

Optional ingredients may be included within the composition of the present invention as long as the physical integrity of the anhydrous solid composition is not compromised and the refractive index of the composition remains between 1.4-1.6.

Pigments are selected from fluorescent, pearlescent and glitter materials. Although the refractive index may not be match the refractive index of the wax or solvent system of the present invention, the pigments of the present invention do not detract from the refractive index of the composition.

Pigments of the present invention are used in an amount of from 0.01 to 10%, preferably from 0.1% to 5%, and most preferably from 0.1% to 1%.

Preferred fragrances are the essential oils. Dermatological agents that may be used in our compositions include vitamins, anti-inflammatory agents, hydroxyacids, and the like, and mixtures thereof. Sunscreens that may be used include dioxybenzone, 2-ethylhexyl 2-cyano-3, 3-diphenylacrylate, 2-ethylhexyl p-methoxycinnamate, 2-ethylhyxyl salicylate, homosalate, menthyl anthranilate, oxybenzone, octyl dimethyl PABA, red petrolatum, titanium dioxide, ferulic acid esters, or mixtures thereof. Preferably the sunscreen is titanium dioxide, ferulic acid esters or mixtures thereof.

The solvent used in compositions of this invention may also comprise other ingredients that are commonly employed by one of skill in the art in compositions for application to the skin (e.g., stabilizers, emollients, microbial agents, antioxidants and the like).

Other biological active agents can be added to the present inventive compositions as desired, as long as the clarity and refractive index are not compromised. Routine experimentation can determine the amounts required to retain a stable composition. The type of biological active agent added, can be any which is beneficially used in a topical cosmetic or pharmaceutical composition. For example, the composition can further comprise moisturizing actives, antibacterials, anti-irritant agents, anti-inflammatory agents, antiaging agents, antiwrinkle agents, sunscreen agents, or vitamins.

The preferred composition is in the form of an anhydrous solid stick composition. This composition is made using a conventional lipstick mold known to those skilled in the art. Specifically, the lipstick is made by first mixing the partially crystalline polyolefin homopolymer or copolymer wax within the solvent system. Any conventional mixing methods may be employed. The composition is then allowed to cool to ambient temperature.

### Method of Use

The present inventive compositions are particularly useful as lipsticks. The composition is administered or topically applied to the lips. It is suggested as an example that topical application range from about once per day to about twice daily. The amount of composition applied to the skin will vary depending upon coverage desired.

The following examples further illustrate the invention, but the invention is not limited thereto.

### EXAMPLE 1

The following composition provides an example of a preferred embodiment incorporating the inventive composition in the form of an anhydrous stick. Additional gellants and emollients may be incorporated, as shown in the composition in Table 1. The examples that follow are conducted utilizing the composition below.

TABLE 1
Composition

| Trade Name | CTFA name | Percent |
|---|---|---|
| Licocene PP 1602 | Polypropylene Wax | 5.000000 |
| Liponate TDTM | Tridecyl Trimellitate | 55.00000 |
| Cab-O-Sil M-5 | Silica | 5.000000 |
| Salacos 120H | Hydroxystearic Acid | 10.000000 |
| Panalane L-14E | Hydrogenated Polyisobutene | 20.00000 |
| Versagel ME 750 | Hydrogenated | 20.00000 |
| | Polyisobutene//ethylene/ | |
| | propylene/styrene | |
| | copolymer//butylenes/ | |
| | ethylene/styrene | |
| | copolymer | |
| Eutanol G | Octyldodecanol | 10.00000 |

### EXAMPLE 2

The following example provides the results of a comparison of four gelatinization agents in combination with eleven solvents, the results of which are provided in Table 2 below. Each composition tested comprises 50% solvent and 50% wax/gelatinization agent.

The physical and chemical stability of the anhydrous solid composition are determined visually. Specifically, the resulting solid is considered to be stable if it has the desired stiffness, as described hereinabove. As a review, the desired stiffness is defined as a solid which does not readily break or crumble and yet is solid enough to retain the oily components of the composition. Clarity is also determined visually.

TABLE 2
Observations: Solvent In Combination With Waxes Waxes/Gelatinization Agent

| | Wax/Gel Agent | | | |
|---|---|---|---|---|
| | LICOCENE PP | LICOCENE PP | LICOCENE PP | |
| | 1302 | 1502 | 1602 | G.P.-1 |
| | (Polypropylene | (Polypropylene | (Polypropylene | (Dibutyl/Laurol |
| Solvent | Wax) | Wax) | Wax) | Glutamide) |
| LUVITOL EHO+ | Slightly opaque, | Slightly opaque, | Slightly opaque, | Frosty, milky, |
| (Cetearyl Octanoate) | unstable solid | unstable solid | stable | yellow |
| | structure | structure | | |
| LIPOVOL MOS+ | Slightly opaque, | Slightly opaque | Slightly opaque, | Frosty, milky, |
| (Tridecyl | Unstable solid | | stable | yellow |
| Stearate/Neopentylgly | structure | | | |
| col Dicaprilate | | | | |
| Dicaprate/Tridecyl | | | | |
| Trimellitate) | | | | |
| HATCOL 200+ | Slightly opaque, | Slightly opaque, | Clear, stable | Frosty, light |
| (Triethylhexyl | stable | unstable sold | | yellow |
| Trimellitate) | | structure | | |
| HATCOL 5103+ | Slightly opaque, | Slightly opaque, | Clear, unable to | Frosty, milky, |
| (Tridecyl Trimellitate) | stable | unstable solid | form solid | yellow |
| | | structure | composition | |
| HATCOL 5131+ | Slightly opaque, | Slightly opaque, | Slightly opaque, | Milky |
| (Isononyl | unstable solid | stable | stable | |
| Isononanoate) | structure | | | |
| CERAPHYL ICA | Slightly opaque, | Slightly opaque, | Slightly opaque, | Frosty, milky |
| (Isocetyl Alcohol) | unstable solid | stable | stable | |
| | structure | | | |
| 99A+ | Frosty/opaque, | Slightly opaque, | Slightly opaque, | Frosty, milky, |
| Isododecane/G.P.1 | unable to form | stable but solid | stable but solid | yellow/shrank |
| | solid composition | shrank | shrank | |
| Silicone 556 Fluid | Slightly opaque, | Slightly opaque, | Slightly opaque, | Frosty, milky, |
| (Phenyl Trimethicone) | stable | unstable solid | stable | yellow |
| | | structure | | |
| EMERY 3004+ | Slightly opaque, | Slightly opaque, | Slightly opaque, | Milky |
| (Polydecene) | stable | stable | stable | |
| PANELANE L-14E+ | Slightly opaque, | Slightly opaque, | Slightly opaque, | Frosty, yellow |
| (Hydrogenated | unstable solid | stable | stable | |
| Polyisobutene) | structure | | | |
| CRODAMOL STS+ | Slightly opaque, | Slightly opaque, | Slightly opaque, | Frosty, yellow |
| (PPG-3 Benzyl Ether | stable | unable to form | unstable | |
| Myristate) | | solid composition | | |

As seen from Table 2 above, the combination of HATCOL 200+ with Licocene PP 1602 yields a clear, stable composition. The other combinations of wax/solvent combinations either fail in producing a clear composition, a stable composition, or both.

What is claimed is:

1. A solid, substantially clear cosmetic composition having a refractive index of between 1.4-1.6 comprising:
a partially crystalline polyolefin homopolymer or copolymer wax; and
a solvent system,
wherein the refractive index of the solvent system is matched with the refractive index of the wax.

2. The composition of claim 1 wherein the partially crystalline polyolefin homopolymer or copolymer wax is polyethylene-propylene copolymer.

3. The composition of claim 1 wherein the solvent system is selected from the group consisting of low polar, organic liquid oil or esters, ethers and hydrocarbons.

4. The composition of claim 1 wherein the solvent system comprises an ester selected from the group consisting of Tridecyl Trimellitate, Isododecane, Isohexadecane, Isoeicosane, Polyisobutene, Isodecyl Neopentanoate, Lauryl Lactate, Ethylhexyl Palmitate, Octyldodecyl Behenate, Isododecyl Ethylhexanoate, Polyglyceryl-2 Diisostearate, Synthetic Jojoba Oil, Octyldodecyl Myristate, Isononyl Isononanoate, Isodecyl Isononanoate, Isotridecyl Isononanoate, Octyl Stearate, Dioctyl Adipate, Dioctyl Adipate/Octyl Palmitate/Octyl Stearate, Octyl Stearate/Octyl Palmitate/Dioctyl Adipate, Hexyl Laurate, Coco-Caprylate/Caprate, Cetearyl Isononanoate, Isocetyl Stearate, Isopropyl Myristate, Isopropyl Palmitate, Propylene Glycol Dipelargonate, Cetyl Octanoate, Tricaprylin, Octyldodecyl Myristate, Isostearyl Neopentanoate, Decyl Oleate, Isodecyl Oleate, Octyldodecyl Stearoyl Stearate, Caprylic/Capric Triglyceride, Hydroxy-terminated Polyurethane, Natural Squalane, Isostearyl Neopentanoate, Octyldodecanol, Oleyl Lactate, Oleyl Alcohol, Oleyl Oleate, Glycereth-7 Triacetate, Polyglyceryl-3 Diisostearate, Pentaerythrityl Tetraisostearate, Triglycerine Diisostearate, Oleyl Lactate, Diglycerol Diisostearate/IPDI Copolymer, Ricinus Communis (Castor) Seed Oil, Menthyl Anthranilate, Ethylhexyl Salicylate, Polyisobutylene, 2-Ethylhexyl-4-methoxycinnamate and combinations thereof.

5. The composition of claim 3 wherein the ester is tridecyl trimellitate.

6. The composition of claim 1 further comprises a gellant selected from the group consisting of Dibutyl/Laurol Glutamide, Steroyl Inulin, Ethyl Hexyl Hydrostearate/Triethylhexyl Trimellitate/C30-45 Olefin, 12-Hydroxystearic Acid, Polyethylene at low concentrations below 1%, Ethylenediamine/Stearyl Dimmer Tallate Copolymer, Polyamide resin, Hydrogenated Styrene/Isoprene Copolymer, Styrene-Ethylene/Butylene-Styrene Black Copolymer, and combinations thereof.

7. A method of making the solid, substantially clear composition of claim 1 comprising the steps of:
(a) mixing the partially crystalline polyolefin homopolymer or copolymer wax within the solvent system; and
(b) cooling said composition to ambient temperature.

8. The method of claim 7 wherein the partially crystalline polyolefin homopolymer or copolymer wax is polyethylene-propylene copolymer.

9. The method of claim 7 wherein the solvent system is selected from the group consisting of low polar, organic liquid oil or esters, ethers and hydrocarbons.

10. The method of claim 7 wherein the solvent system comprises an ester selected from the group consisting of Tridecyl Trimellitate, Isododecane, Isohexadecane, Isoeicosane, Polyisobutene, Isodecyl Neopentanoate, Lauryl Lactate, Ethylhexyl Palmitate, Octyldodecyl Behenate, Isododecyl Ethylhexanoate, Polyglyceryl-2 Diisostearate, Synthetic Jojoba Oil, Octyldodecyl Myristate, Isononyl Isononanoate, Isodecyl Isononanoate, Isotridecyl Isononanoate, Octyl Stearate, Dioctyl Adipate, Dioctyl Adipate/Octyl Palmitate/Octyl Stearate, Octyl Stearate/Octyl Palmitate/Dioctyl Adipate, Hexyl Laurate, Coco-Caprylate/Caprate, Cetearyl Isononanoate, Isocetyl Stearate, Isopropyl Myristate, Isopropyl Palmitate, Propylene Glycol Dipelargonate, Cetyl Octanoate, Tricaprylin, Octyldodecyl Myristate, Isostearyl Neopentanoate, Decyl Oleate, Isodecyl Oleate, Octyldodecyl Stearoyl Stearate, Caprylic/Capric Triglyceride, Hydroxy-terminated Polyurethane, Natural Squalane, Isostearyl Neopentanoate, Octyldodecanol, Oleyl Lactate, Oleyl Alcohol, Oleyl Oleate, Glycereth-7 Triacetate, Polyglyceryl-3 Diisostearate, Pentaerythrityl Tetraisostearate, Triglycerine Diisostearate, Oleyl Lactate, Diglycerol Diisostearate/IPDI Copolymer, Ricinus Communis (Castor) Seed Oil, Menthyl Anthranilate, Ethylhexyl Salicylate, Polyisobutylene, 2-Ethylhexyl-4-methoxycinnamate and combinations thereof.

11. The method of claim 9 wherein the ester is tridecyl trimellitate.

12. The method of claim 7 further comprising a gellant selected from the group consisting of Dibutyl/Laurol Glutamide, Steroyl Inulin, Ethyl Hexyl Hydrostearate/Triethylhexyl Trimellitate/C30-45 Olefin, 12-Hydroxystearic Acid, Polyethylene at low concentrations below 1%, Ethylenediamine/Stearyl Dimmer Tallate Copolymer, Polyamide resin, Hydrogenated Styrene/Isoprene Copolymer, Styrene-Ethylene/Butylene-Styrene Black Copolymer, and combinations thereof.
